(19) United States
(12) Patent Application Publication
(10) Pub. No.: US 2022/0155254 A1
(43) Pub. Date: May 19, 2022

(54) Seawater Alkalinity Detection System

(71) Applicant: Tien-I Bao, Taoyuan City (TW)

(72) Inventor: Tien-I Bao, Taoyuan City (TW)

(21) Appl. No.: 16/951,751

(22) Filed: Nov. 18, 2020

(51) Int. Cl. G01N 27/416; G01N 33/18; G01F 23/00; G01N 1/14

(52) U.S. Cl. CPC G01N 27/4167; G01N 1/14; G01F 23/00; G01N 33/18

(57) ABSTRACT

A seawater alkalinity detection system includes a mainframe, a container, and an external apparatus. The mainframe includes a pump motor, a PH value detecting pole, a dosing delivery port, a seawater delivery port, a KH to PH converter output port, a power supply port, a network connection control unit, a detecting operation unit, and a water level controller. The container defines a first detection space containing a contrast liquid, a second detection space containing a reference liquid, and a receiving space accommodating the contrast liquid that is tested. Thus, the seawater alkalinity detection system that automatically detects the KH value of the reference liquid in the seawater storage bucket, and automatically controls the dosing data to add the additive automatically to change the second PH value of the reference liquid in the seawater storage bucket, without having to add a medicinal liquid.

### BACKGROUND OF THE INVENTION

### 1. Field of the Invention

The present invention relates to a detection system and, more particularly, to a seawater alkalinity detection (or testing) system.

### 2. Description of the Related Art

In the process of raising corals and marine fish, the alkaline value or alkalinity is very important. The keepers must regularly check the alkalinity (KH) value in the seawater to maintain the alkalinity at a predetermined value so as to keep the fish safe. An acid-base indicator dosing or titration is a method for testing the KH value in the seawater. In a conventional acid-base indicator dosing method, a determined amount of acid solution is dripped into a tested solution to calculate the KH value in the tested solution by variation of the PH value. However, the acid solution will chemically change the elements in the tested solution so that the tested solution cannot be returned to the feeding tank for reuse. In another conventional acid-base indicator dosing method, air is infused into a control solution and a tested solution, wherein the control solution has a known KH value. After the carbon dioxide (CO2) concentration in the control solution and the tested solution reaches a steady state, the operator measures the PH value of the control solution and the tested solution, to calculate the KH value of the tested solution according to the differential of the control solution and the tested solution. In such a manner, the control solution and the tested solution can be returned to the feeding tank for reuse. However, this method must be tested and compared by a manual labor, thereby causing inconvenience to the user.

### BRIEF SUMMARY OF THE INVENTION

The primary objective of the present invention is to provide a seawater alkalinity detection system that automatically detects the KH value of the reference liquid in the seawater storage bucket, and automatically controls and presets the dosing data to add the additive automatically to change the second PH value of the reference liquid in the seawater storage bucket, without having to add a medicinal liquid.

In accordance with the present invention, there is provided a seawater alkalinity detection system comprising a mainframe and a container located beside the mainframe. The mainframe includes a pump motor, a PH value detecting pole, a dosing delivery port, a seawater delivery port, a KH to PH converter output port, a power supply port, a network connection control unit, and a detecting operation unit. The pump motor is provided with two pumping tubes. The seawater delivery port is connected to a seawater storage bucket. The power supply port is connected to an external power supply. The container defines a first detection space, a second detection space, and a receiving space. The first detection space contains a contrast liquid therein. The second detection space contains a reference liquid therein. The reference liquid is supplied by the seawater delivery port. The receiving space accommodates the contrast liquid that is tested. The two pumping tubes extend into and are connected to the first detection space and the second detection space to pump air into the first detection space and the second detection space respectively. The PH value detecting pole is disposed in the first detection space to test the contrast liquid that is pumped, to derive a first PH value. The contrast liquid that is tested is delivered from the first detection space to the receiving space. The reference liquid that is pumped is delivered from the second detection space to the first detection space, and the PH value detecting pole tests the reference liquid, to derive a second PH value. The detecting operation unit analyzes the first PH value and the second PH value to derive an alkalinity (KH) value of the reference liquid. An additive is added into the reference liquid in the seawater storage bucket according to the KH value to change the second PH value of the reference liquid in the seawater storage bucket. The additive is delivered through the dosing delivery port into the reference liquid. The KH to PH converter output port transfers the KH value into a corresponding PH value and outputs the corresponding PH value.

Further benefits and advantages of the present invention will become apparent after a careful reading of the detailed description with appropriate reference to the accompanying drawings.

### BRIEF DESCRIPTION OF THE SEVERAL VIEWS OF THE DRAWING(S)

FIG. 1 is a perspective view of a seawater alkalinity detection system in accordance with the preferred embodiment of the present invention.

FIG. 2 is a partial exploded perspective view of the seawater alkalinity detection system in accordance with the preferred embodiment of the present invention.

FIG. 3 is a block diagram of an application program of the seawater alkalinity detection system in accordance with the preferred embodiment of the present invention.

FIG. 4 is a schematic operational view showing the contrast liquid and the reference liquid are pumped.

FIG. 5 is a schematic operational view showing the contrast liquid is tested.

FIG. 6 is a schematic operational view showing the reference liquid is tested.

### DETAILED DESCRIPTION OF THE INVENTION

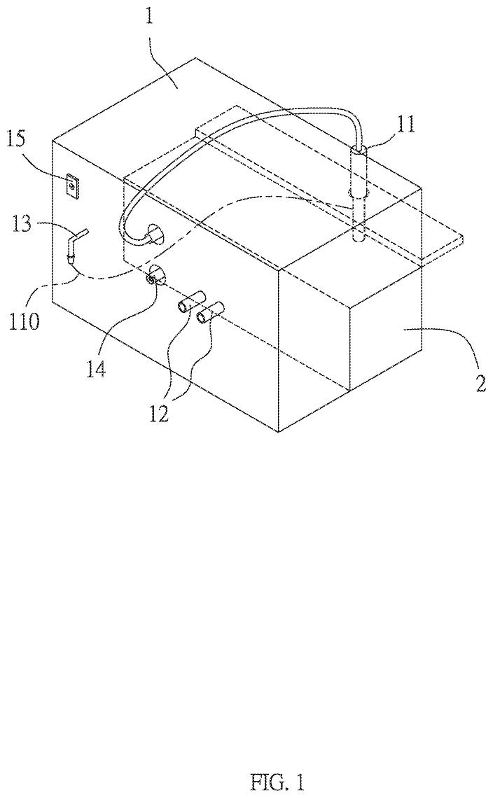

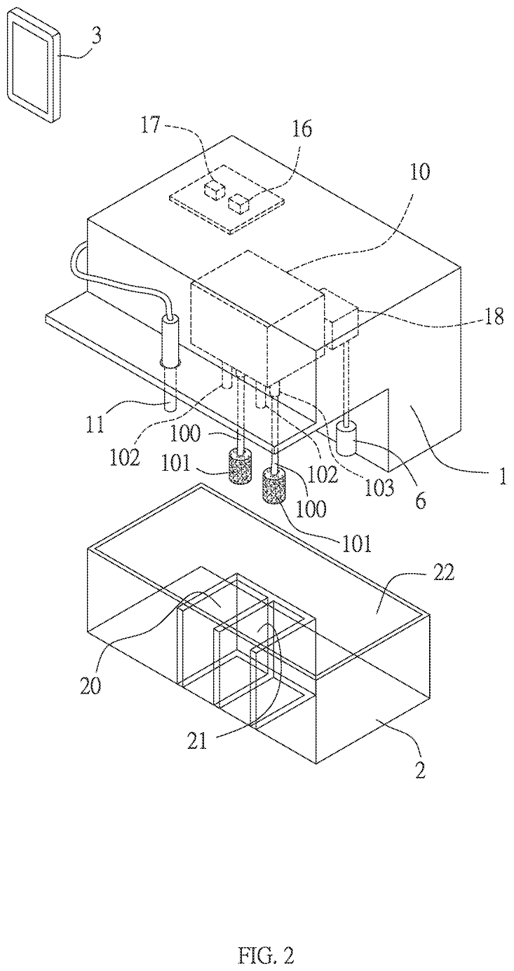

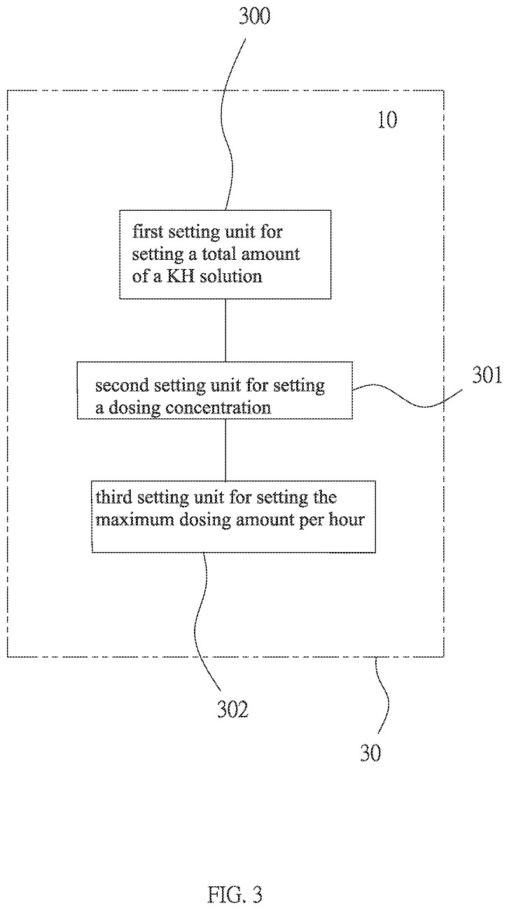

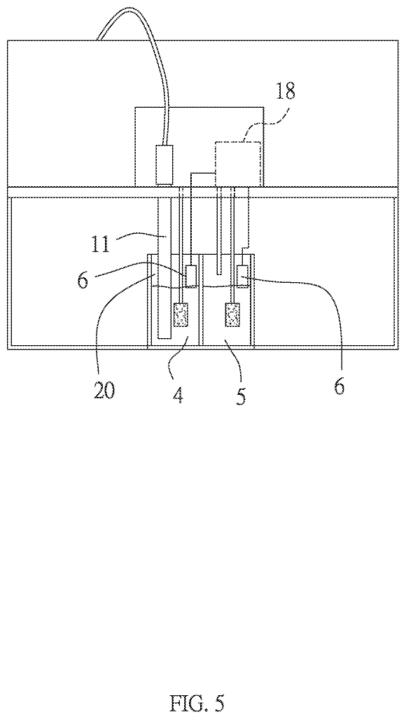

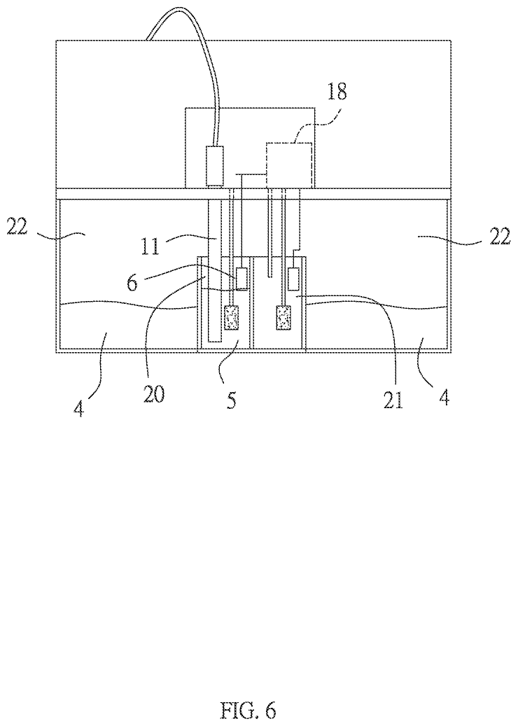

Referring to FIGS. 1-6, a seawater alkalinity detection system in accordance with the preferred embodiment of the present invention comprises a mainframe 1 and a container 2 located beside the mainframe 1.

The mainframe 1 includes a pump motor 10, a PH value detecting pole 11, a dosing delivery port 12, a seawater delivery port 13, a KH to PH converter output port 14, a power supply port 15, a network connection control unit 16, and a detecting (or testing) operation unit 17. The pump motor 10 is provided with two pumping tubes 100. The seawater delivery port 13 is connected to a seawater storage bucket (or tank). The power supply port 15 is connected to an external power supply.

The container 2 defines a first detection space 20, a second detection space 21, and a receiving space 22. The first detection space 20 contains a contrast (or control) liquid 4 therein. The second detection space 21 contains a reference liquid 5 therein. The reference liquid 5 is supplied by the seawater delivery port 13. The receiving space 22 accommodates the contrast liquid 4 that is tested.

The two pumping tubes 100 extend into and are connected to the first detection space 20 and the second detection space 21 to pump air into the first detection space 20 and the second detection space 21 respectively. The PH value detecting pole 11 is disposed in the first detection space 20 to test the contrast liquid 4 that is pumped, to derive a first PH value. The contrast liquid 4 that is tested is delivered from the first detection space 20 to the receiving space 22. The reference liquid 5 that is pumped is delivered from the second detection space 21 to the first detection space 20, and the PH value detecting pole 11 tests the reference liquid 5, to derive a second PH value.

The detecting operation unit 17 analyzes the first PH value and the second PH value to derive an alkalinity (KH) value of the reference liquid 5. In practice, the PH value is an analogue signal, and the KH value is a digital signal. The PH electrodes output an analogue voltage signal which is transferred into a digital signal by an ADC, and the digital signal is sent to the detecting operation unit 17. An additive (such as soda water) is added into the reference liquid 5 in the seawater storage bucket according to the KH value to change the second PH value of the reference liquid 5 in the seawater storage bucket. The additive is delivered through the dosing delivery port 12 into the reference liquid 5. The additive is added by dosing. The KH to PH converter output port 14 transfers the KH value into a corresponding PH value and outputs the corresponding PH value.

In the preferred embodiment of the present invention, the seawater alkalinity detection system further comprises an external apparatus 3 (such as a smart phone or a computer) including an application program (APP) 30 connected to the network connection control unit 16. The network connection control unit 16 is connected to an external network and is connected to the APP 30 of the smart phone, so as to transmit messages, such as upload testing results and download parameters. The APP 30 includes a first setting unit 300, a second setting unit 301, and a third setting unit 302. The first setting unit 300 presets a total amount of a KH solution. The second setting unit 301 presets a dosing concentration. The third setting unit 302 presets the maximum dosing amount per hour.

In the preferred embodiment of the present invention, the mainframe 1 further includes a noise eliminating member 110 mounted on the PH value detecting pole 11 and extending to the seawater delivery port 13.

In the preferred embodiment of the present invention, each of the two pumping tubes 100 has an end provided with a pumping stone (or aerator) 101. Thus, the pumping stone 101 changes the density of the air bubbles and makes the air bubbles denser to enhance the air pumping effect.

In the preferred embodiment of the present invention, the pump motor 10 is provided with at least one air suction port 102 and a plurality of air outlet ports 103. The two pumping tubes 100 are inserted into the air outlet ports 103. A predetermined distance is defined between the at least one air suction port 102 and the first detection space 20 and the second detection space 21.

In the preferred embodiment of the present invention, the predetermined distance is smaller than or equal to 2 cm (centimeter).

In the preferred embodiment of the present invention, each of the air outlet ports 103 has an air output rate greater than 1 L/m (liter per minute).

In the preferred embodiment of the present invention, the contrast liquid 4 is reference seawater.

In the preferred embodiment of the present invention, the reference liquid 5 is seawater in the seawater storage bucket.

In the preferred embodiment of the present invention, the mainframe 1 further includes a water level controller 18 and a water level detector 6 (such as a float or a capacitor) for starting the water level controller 18. The water level controller 18 delivers the contrast liquid 4 that is tested from the first detection space 20 to the receiving space 22, and delivers the reference liquid 5 that is pumped from the second detection space 21 to the first detection space 20.

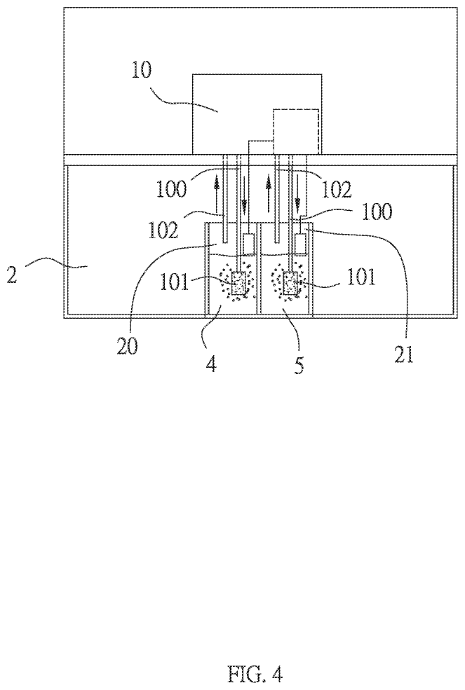

In operation, referring to FIGS. 4-6 with reference to FIGS. 1-3, the contrast liquid 4 is added into the first detection space 20 of the container 2, and the reference liquid 5 of the seawater storage bucket is delivered through the seawater delivery port 13 into the second detection space 21 of the container 2. At this time, the reference liquid 5 is seawater that has been used in the seawater storage bucket, and the contrast liquid 4 is seawater that has not been used. After the water level controller 18 is started by the water level detector 6, the water level controller 18 sends a signal to a CPU. The water of the second detection space 21 is drawn to the first detection space 20 when the receiving space 22 lacks water and needs replenishment. The CPU draws the water of the first detection space 20 into the receiving space 22 completely, and draws the water of the second detection space 21 into the seawater storage bucket completely.

The water of the first detection space 20 is drawn to the second detection space 21 when the water of the receiving space 22 is too much. The CPU draws the water of the first detection space 20 into the receiving space 22 completely, and draws the water of the second detection space 21 into the seawater storage bucket completely.

Then, the two pumping tubes 100 pump air into the first detection space 20 and the second detection space 21 to pump the contrast liquid 4 and the reference liquid 5 respectively. The at least one air suction port 102 and the air outlet ports 103 form an air circulation.

Then, the PH value detecting pole 11 performs a PH value test on the contrast liquid 4 that is pumped, to derive the first PH value. Then, the water level controller 18 is started by the water level detector 6 to deliver the contrast liquid 4 that is tested from the first detection space 20 to the receiving space 22, and to deliver the reference liquid 5 that is pumped from the second detection space 21 to the first detection space 20. The PH value detecting pole 11 performs a PH value test on the reference liquid 5, to derive the second PH value. Finally, the detecting operation unit 17 analyzes the first PH value and the second PH value to derive the alkalinity (KH) value of the reference liquid 5. The information is transmitted through the network to the website.

The APP 30 of the external apparatus 3 is connected to the network connection control unit 16 of the mainframe 1 to control and preset the dosing conditions of the additive. The dosing conditions are preset by the first setting unit 300, the second setting unit 301, and the third setting unit 302. The first setting unit 300, the second setting unit 301, and the third setting unit 302 contain preset values that are adjusted according to the practical seawater condition of the seawater storage bucket.

The first setting unit 300 presets the total amount of a KH solution, the second setting unit 301 presets the dosing concentration, and the third setting unit 302 presets the maximum dosing amount (or volume) per hour. The total amount of a KH solution is the volume of a bucket of soda water. The dosing concentration is the dosing volume (ml) that is needed for increasing 1 dKH of soda water. For example, the total amount of the soda water is 1,000 ml. The dosing concentration is 200 ml/dKH. The maximum dosing amount per hour is 0.1 dKH/hr. By calculation, 0.1 dKH/hr*200 ml/dKH is equal to 20 ml/hr. Thus, the maximum dosing amount is 20 ml.

The amount of the additive (such as the soda water) is replenished according to the data preset by the first setting unit 300, the second setting unit 301, and the third setting unit 302 of the APP 30. Thus, the additive is delivered through the dosing delivery port 12 and added into the reference liquid 5 in the seawater storage bucket according to the derived KH value so as to change the second PH value of the reference liquid 5 in the seawater storage bucket.

In addition, the PH value is an analogue signal, and the KH value is a digital signal so that the KH value has to be converted into a PH value that can be transmitted to the external apparatus 3. Thus, the KH to PH converter output port 14 transfers the KH value into a corresponding PH value and outputs the corresponding PH value. For example, the analogue signal of the PH value is about −170 mv to 170mv, and the digital signal of the KH value is 7.5. Thus, the detecting operation unit 17 converts the number into the analogue signal of the PH value 7.5 that is read by an external PH monitor.

It is clear that, the APP 30 of the external apparatus 3 controls and presets the dosing data to add the additive automatically to change the second PH value of the reference liquid 5 in the seawater storage bucket, so as to automatically detect the KH value of the seawater storage bucket without having to add a medicinal liquid.

It is appreciated that, the noise eliminating member 110 is mounted on the PH value detecting pole 11 and extends to the seawater delivery port 13. Thus, noise existing in the seawater of the seawater storage bucket is delivered through the noise eliminating member 110 and conducted to the PH value detecting pole 11. At this time, the PH value detecting pole 11 has a grounding effect to eliminate the noise directly.

Accordingly, the seawater alkalinity detection system automatically detects the KH value of the reference liquid 5 in the seawater storage bucket, and automatically controls and presets the dosing data to add the additive automatically to change the second PH value of the reference liquid 5 in the seawater storage bucket, without having to add a medicinal liquid.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the scope of the invention.

1. A seawater alkalinity detection system comprising:
a mainframe; and
a container located beside the mainframe;
wherein:
the mainframe includes a pump motor, a PH value detecting pole, a dosing delivery port, a seawater delivery port, a KH to PH converter output port, a power supply port, a network connection control unit, and a detecting operation unit;
the pump motor is provided with two pumping tubes;
the seawater delivery port is connected to a seawater storage bucket;
the power supply port is connected to an external power supply;
the container defines a first detection space, a second detection space, and a receiving space;
the first detection space contains a contrast liquid therein;
the second detection space contains a reference liquid therein;
the reference liquid is supplied by the seawater delivery port;
the receiving space accommodates the contrast liquid that is tested;
the two pumping tubes extend into and are connected to the first detection space and the second detection space to pump air into the first detection space and the second detection space respectively;
the PH value detecting pole is disposed in the first detection space to test the contrast liquid that is pumped, to derive a first PH value;
the contrast liquid that is tested is delivered from the first detection space to the receiving space;
the reference liquid that is pumped is delivered from the second detection space to the first detection space, and the PH value detecting pole tests the reference liquid, to derive a second PH value;
the detecting operation unit analyzes the first PH value and the second PH value to derive an alkalinity (KH) value of the reference liquid;
an additive is added into the reference liquid in the seawater storage bucket according to the KH value to change the second PH value of the reference liquid in the seawater storage bucket;
the additive is delivered through the dosing delivery port into the reference liquid; and
the KH to PH converter output port transfers the KH value into a corresponding PH value and outputs the corresponding PH value.

2. The seawater alkalinity detection system as claimed in claim 1, further comprising:
an external apparatus including an application program (APP) connected to the network connection control unit;
wherein:
the APP includes a first setting unit, a second setting unit, and a third setting unit;
the first setting unit presets a total amount of a KH solution;
the second setting unit presets a dosing concentration; and
the third setting unit presets the maximum dosing amount per hour.

3. The seawater alkalinity detection system as claimed in claim 1, wherein the mainframe further includes a noise eliminating member mounted on the PH value detecting pole and extending to the seawater delivery port.

4. The seawater alkalinity detection system as claimed in claim 1, wherein each of the two pumping tubes has an end provided with a pumping stone.

5. The seawater alkalinity detection system as claimed in claim 1, wherein:
the pump motor is provided with at least one air suction port and a plurality of air outlet ports;
the two pumping tubes are inserted into the air outlet ports; and
a predetermined distance is defined between the at least one air suction port and the first detection space and the second detection space.

6. The seawater alkalinity detection system as claimed in claim 5, wherein the predetermined distance is smaller than or equal to 2 cm (centimeter).

7. The seawater alkalinity detection system as claimed in claim 5, wherein each of the air outlet ports has an air output rate greater than 1 L/m (liter per minute).

8. The seawater alkalinity detection system as claimed in claim 1, wherein the contrast liquid is reference seawater.

9. The seawater alkalinity detection system as claimed in claim 1, wherein the reference liquid is seawater in the seawater storage bucket.

10. The seawater alkalinity detection system as claimed in claim 1, wherein:
the mainframe further includes a water level controller and a water level detector for starting the water level controller; and
the water level controller delivers the contrast liquid that is tested from the first detection space to the receiving space, and delivers the reference liquid that is pumped from the second detection space to the first detection space.
